(19) United States
(12) Patent Application Publication
(10) Pub. No.: US 2010/0260690 A1
(43) Pub. Date: Oct. 14, 2010

(54) STABLE CHEWING GUM COMPOSITIONS COMPRISING MALTITOL AND PROVIDING RAPID RELEASE OF NICOTINE

(76) Inventors: Arne Kristensen, Helsingborg (SE); Anders Axelsson, Bjarred (SE); Nils Siegbahn, Angelholm (SE)

Correspondence Address: WOMBLE CARLYLE SANDRIDGE & RICE, PLLC, ATTN: PATENT DOCKETING, P.O. BOX 7037, ATLANTA, GA, 30357-0037, US

(21) Appl. No.: 12/678,780

(22) PCT Filed: Sep. 18, 2008
(86) PCT No.: PCT/EP2008/062462

§ 371 Date: Jun. 18, 2010

(30) Foreign Application Priority Data
Sep. 18, 2007 (DK) PA 2007 01344

(51) Int. Cl. A61K 9/68; A61P 25/34

(52) U.S. Cl. 424/48

(57) ABSTRACT

Use of a nicotine-cellulose combination, maltitol and a gum base for the preparation of a chewing gum composition for achieving a fast onset of nicotine effect after initiation of chewing the chewing gum composition by a subject. The chewing gum composition is preferably prepared by direct compression and it does not disintegrate during chewing. The invention also relates to chewing gum compositions comprising nicotine, which compositions provide a rapid release of nicotine.

### CROSS-REFERENCE TO RELATED APPLICATION

The present application is a National Stage Application of PCT/EP2008/062462, filed Sep. 18, 2008, which claims priority to Denmark Patent Application PA 2007 01344, filed Sep. 18, 2007, each of which is incorporated by reference herein in its entirety.

### FIELD OF THE INVENTION

The present invention relates to the use of a nicotine-cellulose combination for the preparation of a chewing gum composition for achieving a fast onset of nicotine effect after initiation of chewing the chewing gum composition by a subject. The composition contains a specific sugar alcohol, namely maltitol, which has proved to be advantageous compared with other sugar alcohols normally used. The advantages being that a less hygroscopic product is provided with improved storage stability. Accordingly, the product is less sensitive to a humid environment (e.g. an atmosphere with a relative humidity of above 75%) and, accordingly, a package for the product can be chosen more freely as it no longer is needed to pack the chewing gum in a package that is strictly impermeable to humidity. The invention also relates to chewing gum compositions comprising nicotine, which compositions provide a rapid release of nicotine.

### BACKGROUND OF THE INVENTION

Smoking behavior is associated with serious health risks not only to the smoker, but also to the people around him exposed to passive smoke. To quit smoking has therefore been the expert's advice for many years. However, the smoker is addicted to nicotine, which makes quitting quite difficult for most smokers. Other ways of nicotine administration have been employed in the efforts to help smokers quit their unhealthy habit. Several products employing oral or transdermal administration of nicotine are currently available for smokers wanting to quit smoking, such as chewing gums, inhalators, patches or mouth sprays.

As the tobacco itself contains several other toxic compounds other than nicotine, nicotine substitution products are also relevant for individuals who consume their tobacco in other ways than by smoking. Mainly in Scandinavia, particularly in Sweden, tobacco is consumed as chewing tobacco or snuff. The use of nicotine substitution products will spare consumers of chewing tobacco or snuff as well as smokers from the carcinogenic risks derived from tobacco.

In spite of the availability of several nicotine substitution products such as those mentioned above, many individuals addicted to nicotine still find it difficult to quit their consumption of tobacco. The explanation for this is probably a combination of multiple factors, of which two of them relate to the attained concentration of nicotine in the bloodstream and more importantly, the rate by which nicotine reaches the bloodstream and thereby provides the user with the desired effect.

The rate by which nicotine reaches the bloodstream can be limited by the in vitro rate by which nicotine is released from the nicotine substitution product. Accordingly, there is a need for pharmaceutical compositions comprising nicotine with a rapid release of nicotine, e.g. a rapid in vitro and/or in vivo release. Furthermore, rapid release of nicotine minimizes the total content of nicotine necessary in the compositions, which is a benefit in terms of the consumer's total intake of this potentially toxic compound and in terms of manufacturing economy.

### SUMMARY OF THE INVENTION

The present invention is a further development of a chewing gum composition provided by the applicant and described in unpublished patent application PCT/EP2007/002344, which is hereby incorporated in its entirety. Moreover, the compositions specifically mentioned in this application are not subject of the present invention and may be disclaimed, if needed. The present inventors have observed that the compositions described in the above-identified PCT application in some cases may be sensitive to the humidity in the environment, which means that specific precautions may be taken especially during the production process. In order to solve this problem, the inventors have found that use of maltitol as a sweetener instead of e.g. sorbitol, or use of maltitol in combination with other sugar alcohols (however, not sorbitol) leads to a product that is much less sensitive towards the humidity in the surroundings, which in turn also means that it is not necessary to take specific precautions during the production process in order to avoid negative impact from the humidity of the surrounding. Moreover, less sensitivity of the product towards the humidity of the surroundings also leads to another advantage, namely a more flexible choice with respect to packaging of the final product. In those cases, where humidity has a negative impact on the product, the product must either be packaged in containers that do not allow access of water to the product, e.g. such containers must be impermeable to water or specific cartridges with water-sorbing material must be included. Moreover, normally such products have a shelf-life of at the most 2-3 years, whereas it is normally possible to obtain longer shelf-lives when humidity is not a problem. Accordingly, the present inventors have developed a novel chewing gum composition that has a better resistance towards humidity and at the same time it provides a rapid release of nicotine and a rapid increase in the plasma concentration of nicotine upon in vivo use. The composition may be used as a pharmaceutical composition and/or as a tobacco substitute composition.

Thus, the present invention relates to a chewing gum composition comprising a nicotine-cellulose combination, maltitol and a gum base. Furthermore, the invention relates to the use of such a composition for achievement of a fast onset of action of nicotine after application of the chewing gum composition to the oral cavity of a subject.

In the present context the term “nicotine-cellulose combination” is intended to denote a solid material composed of a cellulose which has sorbed (adsorbed and/or absorbed) a well-defined amount of nicotine (either as free base or as a pharmaceutically acceptable salt, complex or solvate) e.g. in and/or onto voids or pores within the cellulose. The terms “nicotine-cellulose adduct” and “nicotine-cellulose carrier complex” as used herein are intended to have the same meaning as the term “nicotine-cellulose combination”. A nicotine-cellulose combination with nicotine sorbed in voids or pores of a cellulose has proved to be much more stable than a mere mixture of nicotine and cellulose. Such nicotine-cellulose combinations are described in WO 04/056363 (NicoNovum AB) wherein especially celluloses with a microcrystalline structure are mentioned (“microcrystalline cellulose”). However, in order to enable sorption in voids and/or pores, a high porosity of the cellulose employed is important. It seems as if a high crystallinity of celluloses often is accompanied by a high porosity. Accordingly and as described herein, the specific origin and nature of the cellulose does not seem to be decisive for the effect, but the porosity of the cellulose is important to achieve the desired sorption in voids and/or pores and, accordingly, to achieve a suitable stability of the nicotine-cellulose combination. As mentioned herein, suitable qualities of cellulose are celluloses having properties (porosity, surface area and/or crystallinity) corresponding to the specific microcrystalline cellulose qualities mentioned (e.g. the Avicel products), but qualities having larger porosity and/or surface area are also comtemplated to be suitable for use in the present context. As used herein cellulose is an example of a carrier.

A composition of the invention has a fast initial release of nicotine, thus, the composition—when subjected to an in vitro release test—within the first 2 minutes after start of the test releases nicotine with a release rate corresponding to 10% w/w or more of the total content in the composition per minute. A suitable dissolution test for chewing gum compositions are described in detail in the Experimental section with reference to PhEur.

Moreover, a chewing gum composition of the invention is non-disintegrating, i.e. it does not disintegrate into particles during chewing of the gum composition, and it does not crumble. It is currently contemplated that use of a particular gum powder as gum base possibly in combination with a suitable selection of additives has impact on the non-disintegrating properties of the chewing gum composition. In specific embodiments, the gum base and/or the chewing gum composition comprises one or more fats, waxes, emulsifiers, plasticizers, oils and/or flavoring agents. Moreover, in a preferred embodiment, the gum base is suitable for direct compression and the chewing gum composition is prepared by direct compression. The chewing gum may be coated or uncoated.

Gum bases having suitable properties and leading to non-disintegrating chewing gum compositions are e.g. gum bases that are or comprise, Gum powder PG 11 TA, Gum powder PG 11 TA New, Gum powder PG 5 TA, Gum powder PG 5 TA New and Gum powder PG N12 TA.

Such gum bases have a content of one or more elastomers, one or more resins, one or more plasticizers, one or more water-insoluble adjuvants and, optionally, one or more antioxidants. The ingredients must be of food grade quality and/or being pharmaceutically acceptable.

In order to achieve a non-disintegrating product and a product with suitable release characteristics, the following composition of the gum base has been found to be suitable:

| | |
|---|---|
| Elastomers | 5-20% w/w |
| Resins | 25-50% w/w |
| Plasticizers | 20-40% w/w |
| Water-insoluble adjuvants | 10-20% w/w |
| Antioxidants | 0-2% w/w |

More specifically, a gum base suitable for use in the present invention has the following composition:

| | |
|---|---|
| Elastomers | 10-20% w/w |
| Resins | 30-45% w/w |
| Plasticizers | 20-35% w/w |
| Water-insoluble adjuvants | 10-20% w/w |
| Antioxidants | 0-1% w/w |

The one or more elastomers are selected from the group consisting of polyisobutylene, polyisoprene, polyvinyl acetate, isobutylene-isoprene copolymers, vinyl acetate-vinyl laurate copolymers and butadiene-styrene copolymers. Other elastomers may also be suitable for use such as those approved by the Food and Drug Administration.

Suitable examples of resins are vegetable resin esters, synthetic resins and/or terpene resins. In the literature, resins are often denoted elastomer plasticizers to which group also ester gums belongs.

The plasticizers as mentioned above (i.e. without the notation “elastomer plasticizers”) are also denoted “softeners”, “emulsifiers” and/or “waxes” in the literature and encompass triglycerides of non-hydrogenated, partially hydrogenated and fully hydrogenated vegetable oil. The fatty acid component of the plasticizers may be fatty acid having a carbon chain length of from C8-C22. Moreover, a plasticizer may be a mono-, di-, or triglyceride of such fatty acids or glycerol esters of low molecular weight carboxylic acids. More details regarding suitable plasticizers are given in WO 03/084338 and WO 2005/004621 (both in the name of Gum Base Co. S.P.A.) that hereby are incorporated by reference. Moreover, details regarding the prepartion of suitable gum bases are also given in these references.

The water-insoluble adjuvant may be any suitable adjuvant such as, e.g., one or more inert mineral fillers (e.g. calcium carbonate, magnesium carbonate, talc, silica, tricalcium phosphate and the like), anti-agglutination agents,

The antioxidant may be any suitable antioxidant approved for food or pharmaceutical use including butylated hydroxyanisole (BHA), butylated hydroxytoluen (BHT), tocopherol and derivatives thereof, propyl gallate and the like.

The above-mentioned gum bases are the main ingredient in gum powder. Gum powder for use in the present context is free flowing, non-adherent gum powder containing 30-50% of the gum base together with food-grade or pharmaceutically acceptable excipients. As the gum powder together with nicotine and additives preferably is manufactured into tablets by direct compression, the excipients employed are typically those normally employed in the preparation of tablets. Such excipients include fillers including cellulose and cellulose derivatives (e.g. hydroxypropylmethylcellulose, hydroxypropylcellulose, microcrystalline cellulose etc), binders (including povidone), glidants or lubricating agents including talc, magnesium stearate and colloidal silica. Such agents are known to a person skilled in the art and reference is given to Remington's Pharmaceutical Sciences. Moreover, aroma and flavours may be added. The gum powder also contains the maltitol. The concentration of maltitol in the gum powder is typically from about 10% to about 30% w/w such as from about 15% to about 25% w/w. Maltitol or any other sugar alcohol (apart from sorbitol) may further be added to the gum powder to prepare the final formulation to be subjected to direct compression,

Normally, the gum base is employed in powdered form and has a mean particle size of about 1 mm (as determined by sieving) or less, such as, e.g., about 0.9 mm or less, about 0.8 mm or less, about 0.7 mm or less, about 0.6 mm or less or about 0.5 mm or less.

A fast onset of the nicotine effect is very important in order to be an acceptable product for the consumer. Accordingly, for a chewing gum composition of the invention, the onset takes place within 3 minutes such as, e.g., within 2.5 minutes or within 2 minutes after application of the chewing gum composition to the oral cavity of the subject. In the present context the term “application to the oral cavity” includes initiation of chewing the chewing gum composition.

Accordingly, in another aspect, the invention relates to a composition in solid or semi-solid dosage form, notably a chewing gum composition, comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 10% w/w or more of the total content in the composition per minute.

As it appears from the examples herein, the present inventors have found that compositions in the form of direct compressed chewing gums are especially suitable to achieve a fast release and a subsequent fast appearance of nicotine in the plasma upon in vivo use. Accordingly, in specific embodiments the invention relates to

i) a direct compressed chewing gum comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 10% w/w or more of the total content in the composition per minute.

Furthermore, the present invention provides methods for preparation of such compositions, comprising mixing nicotine, or a pharmaceutically acceptable salt or derivative thereof, and one or more pharmaceutical acceptable excipients and forming it into a suitable solid or semi-solid dosage form. In one embodiment of the present invention, the dosage form is a chewing gum comprising nicotine, which is obtained by direct compression (DC) of the chewing gum components. The method for preparation of such DC chewing gum comprises mixing the nicotine-containing compound with a gum powder comprising a gum base and one or more pharmaceutical acceptable excipients and compressing this mixture in a tabletting machine.

The present invention also relates to the use of compositions according to the invention, for treatment of nicotine addiction or nicotine withdrawal symptoms.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 shows the stability at 30° C. and 65% RH of nicotine DC chewing gums according to the invention (see Example 5 for details); and

FIG. 2 shows results of the bioequivalence study in Example 6 with respect to craving.

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

As mentioned above, the present invention relates to nicotine-containing compositions that release nicotine very fast in order to achieve a very fast rise in plasma concentration upon administration, especially by the oral mucosa. In particular, the invention relates to compositions in a form that is suitable for delivering nicotine to the oral mucosa such as chewing gums.

In a first aspect, the invention relates to a chewing gum composition comprising a nicotine-cellulose combination, maltitol and a gum base. An especially preferred gum base is gum powder PG N12 TA from Gumbase S.p.A. However, as mentioned in the following other gum bases may also be employed.

As mentioned above, an important characteristic of the composition of the invention is the content of maltitol in order to improve the stability of the final product. Accordingly, a composition according to the invention contains from about 2% w/w to about 20% w/w of maltitol.

The use of maltitol is envisaged to be a very important individual factor in obtaining a suitable stability for the final product. In a specific embodiment the gum base does not (or at the most 5% w/w) contain polyvinyl acetate thereby avoiding a too hygroscopic product, i.e. a product that is relatively easy to manufacture without specific precautions to avoid negative impact from the humidity in the surrounding atmosphere.

Initiated investigations of the resulting product seem to have a suitable stability towards humidity. Accordingly, in a test, which normally is regarded as a challenge for humidity-sensitive products, namely a stability test in open petri dishes, it is envisaged that the composition of the invention fulfills the following requirements:

i) the weight of the composition must at the most increase by 15% after storage in an open petri dish at 25° C. and a relative humidity of 60% for one week,

ii) the increase in weight is at the most about 10% such as at the most about 7.5%, at the most about 5%, at the most about 2.5%, at the most about 1% or at the most about 0.05% after storage in an open petri dish at 25° C. and a relative humidity of 60% for one week,

iii) the weight of the composition at the most increases by 15% after storage in an open petri dish at 25° C. and a relative humidity of 60% for two weeks,

iv) the increase in weight is at the most about 10% such as at the most about 7.5%, at the most about 5%, at the most about 2.5%, at the most about 1% or at the most about 0.05% after storage in an open petri dish at 25° C. and a relative humidity of 60% for two weeks,

v) the weight of the composition at the most increases by 15% after storage in an open petri dish at 25° C. and a relative humidity of 65% for one week,

vi) the increase in weight is at the most about 10% such as at the most about 7.5%, at the most about 5%, at the most about 2.5%, at the most about 1% or at the most about 0.05% after storage in an open petri dish at 25° C. and a relative humidity of 65% for one week,

vii) the weight of the composition at the most increases by 15% after storage in an open petri dish at 25° C. and a relative humidity of 65% for two weeks, and/or

viii) the increase in weight is at the most about 10% such as at the most about 7.5%, at the most about 5%, at the most about 2.5%, at the most about 1% or at the most about 0.05% after storage in an open petri dish at 25° C. and a relative humidity of 65% for two weeks.

Another stability test carried out at 30° C. and 65% relative humidity and reported in the Examples herein clearly shows a significant improvement in stability when sorbitol is replaced with maltitol. Thus, after 3 months storage in an aluminium pack at the above-mentioned conditions, the product with maltitol (and without sorbitol) had a total concentration of N-oxides of 0.08-0.4%, whereas the product with sorbitol after storage under the same conditions had a total concentration of N-oxides of 2.5% w/w.

In specific embodiments, the present invention provides a chewing gum composition in solid or semi-solid dosage form comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 10% w/w or more of the total content in the composition per minute. As demonstrated in the examples herein, such a fast release is not obtained by marketed compositions in the form of chewing gum such as Nicorette®. To this end, the present inventors have found that especially directly compressed chewing gum offers advantages over the Nicorette® chewing gum compositions and, furthermore, the use of a nicotine-containing compound in a specific form may also be advantageous in order to obtain as fast a release as possible.

More specifically, the above-mentioned release rate within the first 2 minutes after start of the test is 10% w/w or more such as, e.g., 11% w/w or more, 12% w/w or more, 13% w/w or more, 14% w/w or more or 15% w/w or more of the total content in the composition per minute.

In a specific embodiment a composition according to the invention comprises a carrier comprising internal voids or pores. Such voids or pores may at least partially comprise said nicotine. Normally, at least 90% w/w or essentially all of the nicotine is contained in such voids or pores The carrier is typically insoluble in water or has a low solubility in water. Thus, it typically has a solubility in water at room temperature of less than 1% w/w.

A particular suitable carrier for use in a snuff composition of the invention is a cellulose having a surface area and/or a porosity at least such as the specifically mentioned Avicel qualiies of microcrystalline cellulose (“mcc”). Certain specific embodiments may also utilize other forms of carriers, in addition to or including mcc, such as but not limited to fibrous material or carbohydrates including cellulose (including hemicellulose, celluloses with different crystallinities and structures (e.g., varying structures including solid fibers, and addition or including fibers or the like in various structures such as web-like structures and/or other structures), including naturally occurring celluloses including Cladophora sp. Algae cellulose or the like), dextran, agarose, agar, pectin, alginate, xanthan, chitosan, starch (including potato starch, shoti starch) etc. or mixtures thereof.

Nicotine may be present in any suitable form such as, e.g. in the form of the free base form of nicotine or in the form of a suitable salt or complex thereof. Moreover, the nicotine may be present in the form of a carrier complex or a carrier adduct, wherein nicotine is present together with a carrier compound. In a specific embodiment, the carrier compound is a particulate material comprising internal voids throughout the material and the voids at least partially comprises said nicotine. While not intended to be bound by theory, it is believed as of the time of this patent application that nicotine may interact with the carrier (for example, mcc or other suitable carrier including other cellulose carriers) by absorbing into and/or adsorbing onto the carrier. Such interaction is completely or nearly completely reversible.

A particular suitable material having internal voids and/or pores is a cellulose such as, e.g., a microcrystalline cellulose. Specific examples of a suitable microcrystalline cellulose is microcrystalline cellulose selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof.

The cellulose may be a synthetic or semi-synthetic cellulose, or it may be derived from natural celluloses.

Suitable carriers may also be those disclosed in WO 2004/064811, which is hereby included by reference.

More specifically, it is contemplated that a relatively high surface area may be of importance for a carrier that is suitable for use. Accordingly, the specific surface area of suitable carriers, notably a cellulose including a microcrystalline cellulose, is normally at least 0.7 m²/g such as, e.g., 1 m²/g. In certain uses, the specific surface area may range between about 0.7 m²/g and at least about 100 m²/g and/or may be anything within this range and/or may be any mixture of sizes within this range. For example, in certain embodiments, the surface area may be from about 0.7 m²/g to about 100 m²/g, from about 1 m²/g to about 100 m²/g, from about 1.5 m²/g to about 100 m²/g, from about 2.0 m²/g to about 100 m²/g, from about 3.0 m²/g to about 100 m²/g, from about 5 m²/g to about 100 m²/g, from about 7 m²/g to about 100 m²/g, from about 10 m²/g to about 100 m²/g, from about 15 m²/g to about 100 m²/g, from about 20 m²/g to about 100 m²/g, from about 25 m²/g to about 100 m²/g, from about 35 m²/g to about 100 m²/g, from about 45 m²/g to about 100 m²/g, from about 50 m²/g to about 100 m²/g, from about 75 m²/g to about 100 m²/g, or any combination of the start and end points in any of the ranges given. For example, in certain embodiments, the surface area may be about 0.7 m²/g, about 1 m²/g, about 1.5 m²/g, about 2.0 m²/g, about 3.0 m²/g, about 5 m²/g, about 7 m²/g, about 10 m²/g, about 15 m²/g, about 20 m²/g, about 25 m²/g, about 35 m²/g, about 45 m²/g, about 50 m²/g, about 75 m²/g, about 100 m²/g and above about 100 m²/g, or combinations thereof. Such carriers having such suitable surface areas may include, but are not limited to, mcc, fibrous material or carbohydrates including cellulose (including hemicellulose, celluloses with different crystallinities and structures (e.g., varying structures including solid fibers, and addition or including fibers or the like in various structures such as web-like structures and/or other structures), including naturally occurring celluloses including Cladophora sp. Algae cellulose or the like), dextran, agarose, agar, pectin, alginate, xanthan, chitosan, starch (including potato starch, shoti starch) etc. and/or mixtures thereof.

The surface area of the cellulose, notably microcrystalline cellulose, is typically at least about 0.7 m²/g, typically in a range of from about 0.7 m²/g to about 1.5 m²/g

Normally, the mean size range of the carrier compound is from about 15 to about 250 μm.

More specifically, in an embodiment of the invention, nicotine is present as a nicotine-cellulose combination in which said nicotine is at least partly (normally essentially fully) sorbed on cellulose and/or is at least partially absorbed into the carrier and/or is at least partially adsorbed onto the carrier (e.g., mcc), or mixtures thereof. Such interaction is completely or nearly completely reversible.

Hence, in certain specific embodiments nicotine is sorbed on microcrystalline cellulose, absorbed into the mcc and/or adsorbed onto the mcc, and/or combinations thereof.

In embodiments of the present invention, the carrier (e.g., but not limited to mcc and/or other naturally-occurring cellulose) is at least partially porous. This porosity may be due, for example but not limited to, the structure of the carrier, for example, branched, fibrous, or weblike structures may have pores. Ranges of pore sizes include but are not limited to pore volumes of about 0.01 cm³/g and include, but are not necessarily limited to pore volume ranges of from about 0.003 cm³/g or less to about 0.025 cm³/g, to about or greater than 0.60 cm³/g.

In general, the nicotine-cellulose combination is present in a composition of the invention in a concentration of at least about 2% w/w such as in a range from about 2% w/w to about 98% w/w, from about 2% to about 96% w/w, from about 2% w/w to about 95% w/w, from about 3% w/w to about 90% w/w, from about 4% w/w to about 85% w/w, from about 5% w/w to about 80% w/w, from about 5% w/w to about 75% w/w, from about 5% w/w to about 70% w/w, or from about 7.5% w/w to about 65% w/w.

In certain embodiments, the amount of nicotine sorbed, for example absorbed into and/or adsorbed onto to carrier can be up to 50% or more of the total weight of the composition. Ranges of the amount of nicotine sorbed onto the carrier in the present invention range for less than about 1% of the total weight of the composition to more than about 50% of the composition, including all amounts within this range. While applicants do not intend the invention to be bound by theory, it is believed at the time of preparing this application that the maximum amount of nicotine that can be sorbed onto and/or into the carrier, thereby affecting the amount, for example the percent nicotine by weight of the total composition (e.g., the maximum percentage) is affected by properties of the carrier, including but not limited to the structure of the carrier, the porosity of the carrier, and the surface area of the carrier.

In specific embodiments, the concentration of the nicotine-cellulose combination in a composition of the invention is present in a concentration such as, e.g., from about 2% w/w (of the total composition) to about 20% w/w, from about 4% w/w to about 19% w/w, from about 5% w/w to about 18% w/w, from about 6% w/w to about 17% w/w, from about 7% w/w to about 16% w/w or from about 8% w/w to about 15% w/w. In particular this is the case in those situations where the dose required of nicotine is relatively small such as, e.g., in up to a 10 mg range.

In an alternative embodiment, the carrier compound is capable of forming a complex with nicotine such as, e.g., in the case that the carrier compound is an ion-exchange compound including polacrilex.

### Concentrations and Amounts of Nicotine

As mentioned above, nicotine may be present in any suitable form. Normally, nicotine is selected from the group consisting of nicotine base, nicotine hydrochloride, nicotine dihydrochloride, nicotine monotartrate, nicotine bitartrate, nicotine sulfate, nicotine zinc chloride such as nicotine zinc chloride monohydrate and nicotine salicylate. In a preferred aspect, nicotine is in its free base form, which easily can be sorbed on a cellulose to form a microcrystalline cellulose-nicotine carrier complex or carrier adduct.

Normally, the nicotine compound (calculated as the free base) is present in a concentration of at least about 0.1% w/w such as in a range from about 0.1% w/w to about 50% w/w such as, e.g., from about 0.5% w/w to about 45% w/w, from about 1.0% w/w to about 40% w/w, from about 1.5% w/w to about 35% w/w, from about 2% w/w to about 30% w/w, from about 2.5% w/w to about 25% w/w, from about 2.5% w/w to about 20% w/w, from about 3% w/w to about 15% w/w.

Especially in compositions containing a relatively small amount of nicotine (e.g. chewing gums), the concentration of the nicotine compound (calculated as the free base) is normally in a range from about 0.1% w/w to about 15% w/w such as, e.g., from about 0.1% w/w to about 14% w/w, from about 0.1% w/w to about 13% w/w, from about 0.1% w/w to about 12% w/w, from about 0.1% w/w to about 11% w/w, from about 0.1% w/w to about 10% w/w as calculated as free nicotine base.

As mentioned above, the nicotine is present in the form of a nicotine-cellulose combination. In general, this combination is present in a concentration of from about 5% to about 100% such as, e.g., from about 10 to about 100%, from about 5% to about 50% or, alternatively, from about 45% to about 100%. The choice of suitable concentration depends on the load of nicotine in the nicotine-cellulose combination and the dosage of nicotine. If the load is relatively high, then the concentration of the combination may be lower, than if the load is relatively low and vice versa. In a specific embodiment using e.g. Avicel® or a similar cellulose quality a concentration of the combination is generally from about 80% w/w to about 98% w/w, such as, e.g., from about 85% w/w to about 98% w/w, from about 90% w/w to about 98% w/w, from about 92% w/w to about 98% w/w, from about 93% w/w to about 97% w/w or from about 94% w/w to about 96% w/w.

The concentration of nicotine (or the pharmaceutically acceptable salt, complex or solvate thereof) in the combination is at the most 70% w/w such as, e.g., at the most 60% w/w, at the most 50% w/w, at the most 45% w/w. The content of nicotine must not be so high that the combination (which is in powder form) “sweats”, so that nicotine desorbs, evaporates or otherwise disappears from the combination. Accordingly, the load of nicotine in the combination is dependent on the particular cellulose employed. If the surface area of the cellulose material is relatively high, then a larger amount of nicotine can be contained therein in a stable manner during a suitable period of time, whereas a cellulose having a smaller surface area normally is indicative for a lower capacity to load nicotine in a suitable manner with respect to stability.

For most cellulose qualities, the concentration of nicotine in the nicotine-cellulose combination is at the most about 45% w/w, such as, e.g., at the most about 40% w/w, at the most about 35% w/w, at the most about 30% w/w, at the most about 25% w/w, at the most about 20% w/w, at the most about 15% w/w, at the most about 12.5% w/w, at the most about 10% w/w, at the most about 9.5% w/w, at the most about 9% w/w, at the most about 8.5% w/w or at the most about 8% w/w, and the concentration being calculated as the nicotine base.

In a specific embodiment, a particulate material (i.e. a nicotine-cellulose combination) according to the present invention has a concentration of nicotine or the pharmaceutically acceptable salt, complex or solvate thereof in the particulate material is at the most about 7.5% w/w such as, e.g., at the most about 7% w/w, at the most about 6.5% w/w, at the most about 6% w/w, at the most about 5.5% w/w, at the most about 5% w/w, at the most about 4.5% w/w, at the most about 4% w/w, at the most about 3% w/w, at the most about 2% w/w or at the most about 1% w/w, and the concentration being calculated as the nicotine base.

In a specific embodiment, a particulate material according to the present invention has a concentration of nicotine or the pharmaceutically acceptable salt, complex or solvate thereof in the particulate material (carrier, notably a cellulose carrier such as the nicotine-cellulose combination) of from about 1% w/w to about 45% w/w, such as, e.g., from about 1% w/w to about 40% w/w, from about 1% w/w to about 35% w/w, from about 2% w/w to about 30% w/w, from about 2% w/w to about 25% w/w, from about 3% w/w to about 20% w/w, from about 3% w/w to about 15% w/w, from about 3% w/w to about 12.5% w/w, from about 3% w/w to about 10% w/w, from about 3% w/w to about 9.5% w/w, from about 3% w/w to about 9% w/w, from about 3% w/w to about 8.5% w/w or from about 3% w/w to about 8% w/w including any combination of start and end point of the mentioned ranges, and the concentration being calculated as the nicotine base.

The amount of the nicotine compound (calculated as the free base) in a composition of the inventions is generally from about 0.5 mg to about 10 mg such as, e.g., from about 1 mg to about 8 mg, from about 1.5 mg to about 7.5 mg, from about 2 mg to about 5 mg, from about 2.5 mg to about 5 mg, from about 3 to about 10 mg, from about 3 to about 7.5 mg or from about 3 mg to about 5 mg such as, e.g., about 1.5 mg, about 2 mg, about 2.5 mg, about 3 mg, about 3.5 mg, about 4 mg, about 5 mg or about 6 mg, as calculated as free nicotine base. In particular a dosage of 2 mg, 3 mg, 4 mg and 6 mg is of commercial interest.

### Buffering Agents

A composition according to the invention may also contain one or more buffering agents. It is generally known that a slightly alkaline reaction (between 7 and 8) in the oral cavity enhances the absorption of nicotine. Accordingly, it may be and advantage to incorporate a buffer substance in the composition such that a slightly alkaline reaction is provided. Especially compositions for release of the nicotine in the oral cavity can advantageously contain a buffer substance, i.e. compositions like chewing gums, lozenges and snuff compositions.

Suitable buffering agents are typically those selected from the group consisting of acetates, glycinates, phosphates, glycerophosphates, citrates such as citrates of alkaline metals, carbonates, hydrogen carbonates, and borates, and mixtures thereof.

If present the one or more buffering agents are present in a concentration from about 0.5% w/w to about 5% w/w, such as, e.g., from about 0.75% w/w to about 4%, w/w, from about 0.75% w/w to about 3%, w/w or from about 1% w/w to about 2%, w/w.

### Sweeteners

Maltitol is employed in a composition of the invention, notably in the gum powder, to increase the stability as well as the sensory properties of the composition. Moreover, substitution of sorbitol by maltitol has been shown to increase the storage stability of a nicotine-containing chewing gum and accordingly, maltitol is a mandatory ingredient in a composition of the invention. Maltitol is normally present in a concentration of at least about 0.05% such as, e.g. from about 0.075% w/w to about 5% w/w or from about 5% to about 35% w/w, such as, e.g., from about 10% w/w to about 35% w/w, from about 15% w/w to about 35% w/w or from about 20% w/w to about 30% w/w based on the final chewing gum composition. The concentration of maltitol in the gum powder is described herein before.

In order to further increase the sensory properties of the composition according to the invention one or more sweeteners may be added, such as sugar alcohols including xylitol, inositol and/or isomalt, or artificial sweeteners such as e.g. aspartame, acesulfame or saccharin.

The concentration of the one or more sweeteners, if present, is normally at least about 0.05% such as, e.g. from about 0.075% w/w to about 5% w/w or from about 5% to about 35% w/w, such as, e.g., from about 10% w/w to about 35% w/w, from about 15% w/w to about 35% w/w or from about 20% w/w to about 30% w/w. The upper limit for artificial sweeteners is normally about 5% w/w, whereas the upper limit for sugar alcohols normally is much higher (35% w/w). In the event that more then one sweetener and/or artificial sweetener are present in a composition, the above-mentioned ranges are also valid for the total concentration of such sweeteners.

### Anti-Oxidants

It is well-known that nicotine is subject to oxidation and accordingly, it may be advantageous to incorporate one or more anti-oxidants, such as, e.g., ascorbyl palmitate and/or sodium ascorbate, in a composition according to the invention. Specific examples of antioxidants are also mentioned in connection with the description of suitable gum bases.

The one or more anti-oxidants may be present in a concentration of from about 0.05% w/w to about 0.3% w/w, such as, e.g., from about 0.1% w/w to about 0.25% w/w or from about 0.15% w/w to about 0.2% w/w.

### Flavouring Agents

In order to improve the organoleptic properties of a composition according to the invention, the composition may include one or more flavouring agents, such as, e.g., menthol flavour, eucalyptus, mint flavour and/or L-menthol, normally present (total concentration of flavouring agents) in a concentration of from about 0.5% w/w to about 12% w/w, from about 1% w/w to about 10% w/w, from about 1.5% w/w to about 9% w/w or from about 2% w/w to about 8% w/w.

### Direct Compressed Chewing Gums (DC Gums)

As mentioned above, an important embodiment of the present invention is a direct compressed (“DC”) chewing gum. As demonstrated in the examples herein, the inventors have found that chewing gums that have been prepared by direct compression have a very favorable rapid initial release of nicotine. The marketed product Nicorette® has not been prepared by direct compression and releases nicotine much slower in the initial phase. Accordingly, the present inventors have found a specific and surprising effect by changing the method for preparing a nicotine-containing chewing gum from the traditionally applied, i.e. mixing of raw materials employing the Bakery type of method followed by extrusion, conditioning, rolling, scoring and finally breaking the gum sheets into individual pieces to direct compression.

Importantly, the present inventors have found that it is crucial to employ specific gum bases in DC compressed nicotine-containing chewing gums in order to obtain a rapid release of nicotine from the composition. Gum bases having properties similar to or substantially similar to the gum bases employed in the examples herein are contemplated as qualities that should be chosen when a chewing gum is prepared by DC due to the fact that such gums have more favorable properties with respect to flowability and compressibility, i.e. properties that are important to enable compression of the gum without e.g. adhesion to the apparatus, incorrect dosing of the gum composition etc.

Direct compressed chewing gum is prepared by using a gum base that is suitable for direct compression together with one or more acceptable excipients normally pharmaceutically acceptable excipients. However, as mentioned above, it is important to use a gum base having suitable properties in order to obtain the desired rapid release. The excipients are selected from the group of excipients normally used within the pharmaceutical industry for the preparation of tablets, i.e. excipients like fillers, disintegrants, binders, lubricants etc. To this end, excipients that enable direct compression are preferred. Guidance may be found in Handbook of Pharmaceutical Excipients edited by Rowe, R. C. et al., 4^{th} edition, Pharmaceutical Press, London 2003, which is hereby incorporated by reference.

Suitable fillers include celluloses and cellulose derivatives including microcrystalline cellulose, hydroxypropylcellulose, sodium carboxymethylcellulose etc.; lactose, starches including potato starch, maize starch etc.

Suitable lubricants include stearates including magnesium stearate, talc, colloidal silica dioxide etc.

Information of the properties of the various marketed gum bases can be obtained from the gum base providers. Suitable gum bases for use in chewing gums according to the invention are obtained in the form of a granular gum base. Specific examples include gum bases provided by e.g. Gum Base Company, Fertin, Gumlink, SPI Pharma, Cafosa, Avant-garde, ATP og Addvantech Pharma and suitable gum bases include Gumpowder PG 11 TA, Gumpowder PG 11 TA New, Gumpowder PG 5 TA, Gumpowder PG 5 TA New and Gumpowder PG N12 TA from Gumbase Company. These specific gum bases from Gum Base Company fulfil the criteria with respect to content of individual ingredients as mentioned herein before (e.g. elastomer, resin, plasticizer, water-insoluble adjuvants etc.) and any other gum bases must also fulfil these criteria. Such gum bases may be Pharmagum S, Pharmagum M and Pharmagum C from SPI pharma and gum base (Laim J TW A), notably in combination with one or more of the Gumpowders mentioned above. It is important the only gum bases or combinations of gum bases that lead to non-disintegrating chewing gum compositions are employed and, accordingly, the Pharmagum bases may need to be used in combination with other gum bases.

A gum base for use in chewing gums according to the invention is normally in powder or granulate form and has a mean particle size of about 1 mm (as determined by sieving) or less, such as, e.g., about 0.9 mm or less, about 0.8 mm or less, about 0.7 mm or less, about 0.6 mm or less or about 0.5 mm or less.

The gum base is normally present in the chewing gum of the invention in a concentration of from about 15% w/w to about 50% w/w such as, e.g., from about 20% w/w to about 35% w/w. The gum powder is normally present in a chewing gum of the invention in a concentration of 25% w/w to about 80% w/w, such as, e.g., from about 30% w/w to about 80% w/w, from about 40% w/w to about 80% w/w or from about 50% w/w to about 80% w/w.

In a chewing gum according to the invention the nicotine is normally present in a concentration from about 0.1% w/w to about 10% w/w such as, e.g., from about 0.1% w/w to about 7.5% w/w, from about 0.1% w/w to about 5% w/w, from about 0.1% w/w to about 2.5% w/w, from about 0.1% w/w to about 1.5% w/w, from about 0.1% w/w to about 1% w/w, from about 0.12% w/w to about 0.8% w/w, from about 0.14% w/w to about 0.6% w/w or from about 0.15% w/w to about 0.4% w/w as calculated as free nicotine base.

More specifically, the nicotine is normally present in an amount of from about 0.5 mg to about 10 mg such as, e.g., from about 1 mg to about 8 mg, from about 1.5 mg to about 7.5 mg, from about 2 mg to about 5 mg, from about 2.5 mg to about 5 mg, from about 3 to about 10 mg, from about 3 to about 7.5 mg or from about 3 mg to about 5 mg as calculated as free nicotine base.

In the preparation of a chewing gum composition according to the invention, maltitol may either be admixed with the gum base or be a part of the gum base composition or it may be admixed with the nicotine-cellulose combination together with the gum base.

In specific embodiments a chewing gum contains 1.5 mg of the nicotine calculated as free nicotine base. The amount 1.5 mg is lower than the marketed Nicorette® chewing gum that contains 2 mg of nicotine. The lowering of the amount of nicotine is due to the observation that a chewing gum according to the invention releases nicotine in such a suitable manner that bioequivalence with respect to AUC is obtained from a 1.5 mg chewing gum when compared with Nicorette® 2 mg. Accordingly, a chewing gum according to the invention has a markedly improved bioavailability of nicotine; in fact the bioavailability is increased by 30%. This, in turn, leads to a reduction in the amount of nicotine in the chewing gum necessary for obtaining the desired effect.

Accordingly, in a separate aspect, the invention relates to a nicotine-containing chewing gum that has a bioavailability that is improved compared with that of Nicorette® and the improvement expressed as the relative bioavailability calculated by AUC_{0-infinity} (tested composition)/AUC_{0-infinity} (Nicorette®)×100% is at least 120% such as, e.g., at least about 130%, at least about 140% or at least about 150%—provided that the composition and Nicorette® contains the same amount of nicotine calculated as free base.

In specific embodiments a chewing gum according to the invention contains 3 mg or 5 mg of said nicotine calculated as free nicotine base.

Nicotine is present in the form of a nicotine-cellulose combination (a carrier complex or a carrier adduct). The carrier complex is typically a nicotine-microcrystalline cellulose carrier complex as described in WO 2004/05663, which is hereby incorporated by reference. Microcrystalline cellulose contains voids that at least partly are filled with the nicotine. One important advantage is that nicotine free base (i.e. in liquid form) easily can fill the voids.

When nicotine is present as a nicotine-cellulose combination, notably a nicotine-microcrystalline cellulose combination and the cellulose notably has a quality like Avicel® or the like, the concentration of the combination is from about 3% w/w to about 20% w/w, such as, e.g., from about 4% w/w to about 19% w/w, from about 5% w/w to about 18% w/w, from about 6% w/w to about 17% w/w, from about 7% w/w to about 16% w/w or from about 8% w/w to about 15% w/w. Normally, the whole content of nicotine is present in the nicotine-cellulose combination. The content of nicotine in the combination is from about 0.5% w/w to about 50% w/w such as from about 0.5% w/w to about 25% w/w, from about 1% w/w to about 15% w/w, as calculated as free nicotine base. More details are given herein or can be calculated based on the details herein.

Moreover, the inventors have found that when used in the preparation of direct compressed chewing gum, it is advantageous to employ a quality of microcrystalline cellulose that has a mean particle size that is not too low and neither too high such as, e.g., at the most about 500 μm, at the most about 450 μm, at the most about 300 μm, or at the most about 200 μm, or from about 5 to about 500 μm, from 10 to about 500 μm, from 15 to about 500 μm, from about 20 to about 500 μm, from about 30 to about 500 μm, from about 40 to about 500 μm, from about 10 to about 400 μm, from about 20 to about 400 μm, from about 30 to about 400 μM, from about 40 to about 400 μm, from about 30 to about 300 μm, from about 40 to about 300 μm, from about 50 to about 250 μm, from about 50 to about 200 μm or from about 75 to about 200 μm. In specific embodiments the particle size used were about 100 p.m.

As mentioned above, a composition according to the invention may further comprise a pharmaceutically acceptable excipient such as, e.g. a filler, a binder, a lubricant, a buffering agent, a stabilizing agent, a pH adjusting agent, a preservative, a coloring agent, a flavoring agent, a taste-masking agent, a sweetener etc.

In chewing gum composition, a suitable buffering agent is a hydrogen carbonate including alkali metal hydrogen carbonates, or a carbonate including alkaline earth metal carbonates.

If present, sugar alcohols such as, e.g., sorbitol and/or isomalt, may be used in an concentration from about 5% w/w to about 35% w/w, such as, e.g., from about 10% w/w to about 35% w/w, from about 15% w/w to about 35% w/w or from about 20% w/w to about 30% w/w.

As mentioned above, a direct compressed composition according to the invention may further comprise one or more anti-adhesives, lubricants, and/or one or more other pharmaceutically acceptable excipients.

In specific embodiments, the one or more anti-adhesives, lubricants and/or glidants are selected from the group consisting of talc, stearates and salts thereof including magnesium stearate; and silica, and mixtures thereof.

In a specific embodiment, talc is present in a concentration from about 0.5% w/w to about 10% w/w, such as, e.g., from about 1% w/w to about 8% w/w, from about 1.25% w/w to about 6% w/w or from about 1.5% w/w to about 4% w/w, and/or magnesium stearate is present in a concentration from about 0.1% w/w to about 5% w/w, such as, e.g., from about 0.2% w/w to about 4% w/w, from about 0.3% w/w to about 3.5% w/w or from about 0.5% w/w to about 3% w/w, and/or silica is present in a concentration from about 0.1% w/w to about 4% w/w, such as, e.g., from about 0.2% w/w to about 3% w/w, from about 0.3% w/w to about 2% w/w or from about 0.4% w/w to about 1.5% w/w.

In specific embodiments the invention relates to chewing gum compositions according to the present invention, i.e. comprising nicotine-cellulose combination, maltitol and a gum base,

i) wherein the nicotine-containing gum releases at least 7.5% w/w nicotine of the total composition within the first two minutes in the in vitro assay described in Ph.Eur using 20 ml phosphate buffer pH 7.4 and a chewing frequency of 43 cycles per minute in this method,

ii) wherein the nicotine-containing gum releases at least 7.5% w/w nicotine of the total composition within the first two minutes in the in vitro assay described in Ph.Eur using 20 ml phosphate buffer pH 7.4 and a chewing frequency of 43 cycles per minute in this method; and wherein the nicotine-containing gum is made by direct compression.

A direct compression nicotine-containing gum comprising

i) a carrier (including a gum base and maltitol);

ii) nicotine, or a pharmaceutically acceptable salt, solvate, complex or derivative thereof, wherein the direct compression nicotine-containing gum releases at least 7.5% w/w nicotine of the total composition within the first two minutes in the in vitro assay described in Ph.Eur using 20 ml phosphate buffer pH 7.4 and a chewing frequency of 43 cycles per minute in this method.

A nicotine-containing gum comprising

i) a carrier (including a gum base and maltitol);

ii) nicotine, or a pharmaceutically acceptable salt, solvate, complex or derivative thereof, wherein the in vivo uptake by a human, as measured by the content of nicotine in the human's serum, is rapid.

A method of delivering nicotine to an individual comprising the steps of delivering to an individual the nicotine-containing chewing gum as described herein.

A method for making a nicotine-containing gum comprising the steps of:

i) preparing a nicotine-containing composition comprising a carrier (including a gum base and maltitol) and nicotine, or a pharmaceutically acceptable salt, solvate, complex or derivative thereof, wherein the in vivo uptake by a human, as measured by the content of nicotine in the human's serum, is rapid,

ii) directly compressing the nicotine-containing composition to form one or more direct compression gums.

A nicotine-containing chewing gum composition comprising

i) a nicotine-cellulose combination (concentration range: 0.5 to 50% w/w)

ii) a gum base (concentration range: 20-75% w/w)

iii) maltitol (concentration range 5-30% w/w)

iv) a buffering agent (concentration range: 0-10% w/w such as 2-6% w/w)

v) one or more artificial sweeteners (concentration range: 0-2% w/w such as 0.1 to 1% w/w),

vi) one or more flavouring agents (concentration range: 0-10% w/w such as 2-8% w/w), and

vii) one or more pharmaceutically acceptable excipients (e.g. fillers such as fillers with sweetening ability like sugar alcohols) (concentration range: 0-80% w/w such as 10-75% w/w, 15-70% w/w, 20-75% w/w or 25-50% w/w),

the chewing gum optionally being provided with a coating.

All particulars and details mentioned above relating to the chewing gum aspect in general apply mutatis mutandis to the above mentioned specific embodiments.

### Other Aspects

The invention also relates to a method for the preparation of a composition according to the invention. Specific details can be founds in the examples herein and a person skilled in the art will know how to find guidance e.g. from pharmaceutical handbook of how to select suitable excipient and how to prepare such compositions.

In further aspects, the invention relates to the use of a composition according to the invention as a tobacco substitute or for the alleviation of nicotine withdrawal symptoms.

In another aspect the invention, the compositions of the invention is for pharmaceutical use.

The invention is described in more detail in the following figures and non-limiting examples.

The invention is described in more details in the following figures and non-limiting examples.

### Methods

### In Vitro Release Test

The compositions according to the invention must fulfill specific requirements with respect to in vitro release of nicotine. A suitable in vitro test depends on the specific composition in question, i.e. a dissolution test for a chewing gum composition is normally different from a dissolution test for a tablet composition. In general, a person skilled in the art will find guidance as to how to choose a relevant dissolution test for a specific composition in the official monographs such as, e.g., the European Pharmacopoeia. Below is described suitable release or tests in case of chewing gum compositions.

### Chewing Gums

The method and apparatus used were according to Ph. Eur. The chewing apparatus comprises a chewing chamber of 20 mL in which the chewing gum composition is chewed by two horizontal pistons, representing the teeth. The horizontal pistons are capable of rotating around their own axis, which ensures maximum chewing. Together with a third vertical piston (representing the tongue) they work at a constant speed. The pistons are driven by compressed air and their movements are carefully controlled. In more details, the dissolution medium employed was 20 ml phosphate buffer pH=7.4 and a chewing frequency of 43 cycles/min were employed. The dissolution test was run for 45 min. The distance between jaws was 1 mm and the temperature was 37° C.

### EXAMPLES

### Example 1

### Direct Compressed Chewing Gum Compositions A, B, C and D Containing 1.5 Mg of Nicotine

Nicotine was sorbed onto microcrystalline cellulose (MCC) as described in WO 2004/056363. Accordingly, in the present example 2.40 ml nicotine was dissolved in 25 ml ethanol (99.5%). 47.6 g MCC of type PH-102 was loaded into a high-speed mixer and the nicotine was slowly added. After vacuum drying of the obtained wetted mass a fine-grained, white powder of nicotine-microcrystalline cellulose carrier complex was obtained. This was then mixed with the ingredients (except magnesium stearate) stated in the following table in a suitable mixer. Magnesium stearate was sieved and added and the resulting powder mixture compressed into tablets using a tablet press equipped with 17 mm punches. Chewing gum with an average mass of 1.25 g was obtained.

TABLE 1
Gum powder for compositions A, B, C and D

| | A | B | C | D |
|---|---|---|---|---|
| | Concen- | Concen- | Concen- | Concen- |
| | tration | tration | tration | tration |
| Ingredients | (% w/w) | (% w/w) | (% w/w) | (% w/w) |
| Gum powder* from | 39.60 | 39.60 | 40.09 | 39.70 |
| Gumbase | | | | |
| Company | | | | |
| Isomalt (Ph. Eur. | 24.50 | 24.60 | 24.88 | 24.60 |
| curr. ed.) | | | | |
| Talc (Ph. Eur. curr. | 3.20 | 3.20 | 3.70 | 3.70 |
| ed.) | | | | |
| Magnesium | 1.50 | 1.50 | 1.70 | 1.70 |
| stearate (Ph. Eur. | | | | |
| curr. ed.) | | | | |
| Silica, colloidal | 0.80 | 0.80 | 0.90 | 0.90 |
| anhydrous (Ph. | | | | |
| Eur. curr. ed.) | | | | |
| Flavours | 6.64 | 5.67 | 4.67 | 4.76 |

*the gum powder employed was for composition A: Gum powder PG 11 TA, for composition B: Gum powder PG 11 TA New, for composition C: Gum powder PG 5 TA and for composition D: Gum powder PG 5 TA New; all gum powders contain sorbitol

As flavours may e.g. eucalyptus oil, mint flavour, menthol flavour or the like, and mixtures thereof be used,

### Example 2

### In Vitro Release of Nicotine from Directly Compressed Chewing Gum Compositions

The in vitro release of compositions A, B, C and D prepared as described in Example 1 is investigated and compared with the in vitro release of the marketed products Nicorette® and Nicotinell® both of which containing 2 mg of nicotine.

The in vitro dissolution tests are performed as described above for chewing gums Concentrations of nicotine in the dissolution medium are measured by a HPLC method.

Initial results indicate a very fast release of nicotine in accordance with the description herein.

### Example 3

### Buffer Effect on In Vivo Uptake of Nicotine from DC

In vivo studies have indicated that a faster absorption of nicotine from the oral cavity can be obtained by adjusting the pH of the saliva to pH above 7.

The effect of buffer on the in vivo uptake of nicotine was tested in a comparison study wherein the following formulations were administered to the subject. The formulations 1, 2, 3 and 4 had essentially the same ingredients in the same amounts as that of composition A of Example 1 (NB sorbitol has been employed instead of maltitol; however, it is envisaged that substitution of sorbitol with maltitol does not affect the in vivo behaviour). In order to vary the content of nicotine and to include a buffer substance, the content of isomalt was adjusted accordingly.

Formulation 1: 4 mg nicotine, buffered (10 mg carbonate and 10 mg sodium hydrogen carbonate).

Formulation 2: 4 mg nicotine, unbuffered.

Formulation 3: 2 mg nicotine, buffered (10 mg carbonate and 10 mg sodium hydrogencarbonate).

Formulation 4: 2 mg nicotine, unbuffered.

For comparison, Nicorette® 2 mg and 4 mg chewing gum were included.

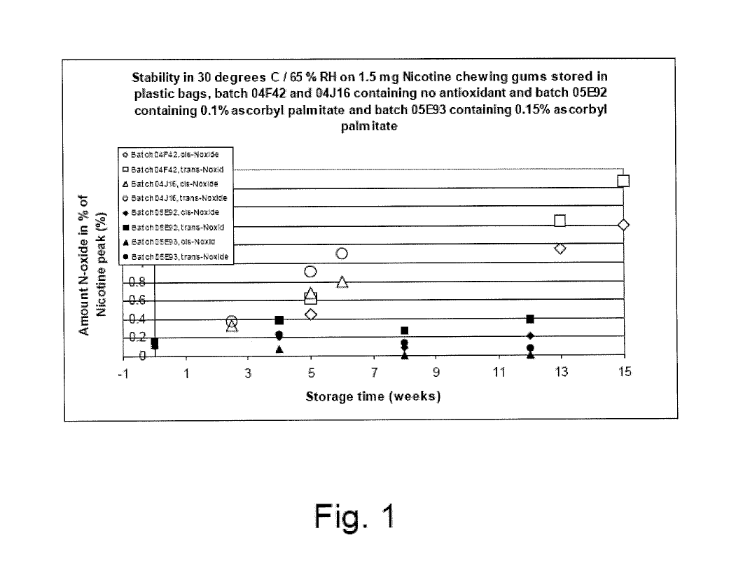

The results are shown in FIG. 1. The results show that the compositions according to the invention have such a fast initial release of nicotine in vitro that even without any buffer substance, they results in in vivo plasma concentrations that are markedly higher than those corresponding to Nicorette® 2 mg or 4 mg, which ever is relevant for comparison purposes. Furthermore, addition of a buffer substance to a composition according to the invention leads to an improved absorption of nicotine. In order words, apart from an initial fast accessibility of nicotine from the compositions according to the invention, a markedly increased absorption of nicotine is seen, i.e. the compositions according to the invention have improved bioavailability (e.g. as measured by AUC or Cₘₐₓ).

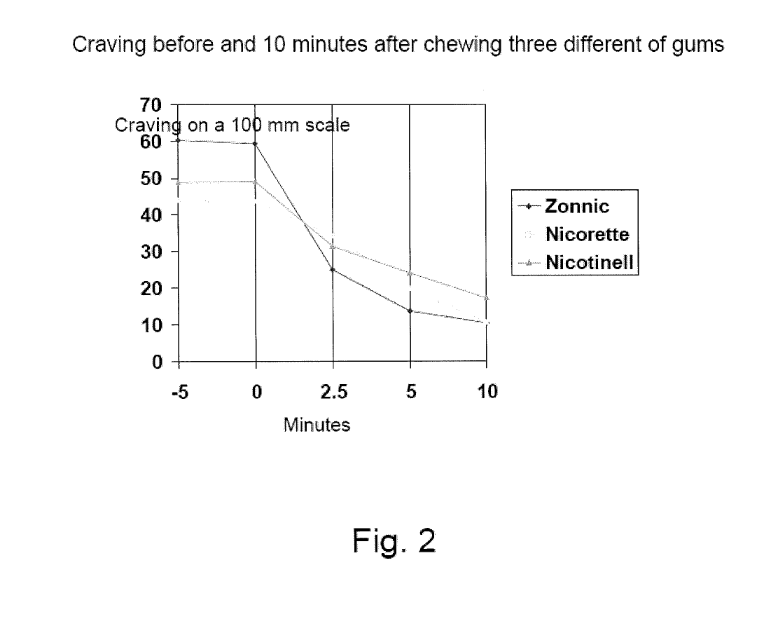

Further studies conducted by the inventors have shown that DC gum without any buffer and containing nicotine in an amount corresponding to 1.5 mg is bioequivalent to Nicorette® chewing gum containing nicotine in an amount corresponding to 2 mg (see FIG. 2.

### Example 4

### DC Gum Compositions Comprising 3 mg Nicotine

Three different chewing gum compositions containing an amount corresponding to 3 mg nicotine were prepared according to Example 1 One composition was without any buffer substance; another contained a buffer substance (i.e. a mixture of sodium carbonate and sodium hydrogen carbonate). The in vivo uptake was measured (n=4) and the results are shown in FIG. 3. FIG. 3 shows that all DC compositions according to the invention perform better than Nicorette® even if the content of nicotine in the DC compositions according to the invention contain 25% less nicotine than Nicorette®.

### Example 5

### Effect of Antioxidants on the Stability of Nicotine

In order to investigate the effect of anti-oxidants on the stability of nicotine in composition, the amount of the nicotine decomposition products cis-N-oxide and trans-N-oxide was measured for DC gums with containing 0%, 0.1% and 0.15% of the anti-oxidant ascorbyl palmitate, respectively. The level of nicotine decomposition products was measured in the compositions after 2.5, 5, 6, 13, 15 and 16 weeks of storage in plastic bags. The amount nicotine decomposition products were determined by reverse phase HPLC.

The result is shown in FIG. 4 and shows that inclusion of anti-oxidant lowers the decomposition of nicotine in the composition.

### Example 6

### In Vitro Release of Chewing Gum Compositions Comprising a Nicotine-Cellulose Combination and Bioequivalence Study

The following chewing gum compositions were prepared by direct compression essentially as described in Example 1.

The chewing gum composition (A) is coated, medicated chewing-gum containing 3 mg nicotine per unit. It is white to off-white, convex, circular shaped with an approximate total weight of 1.575 g, height of 6.3 mm and diameter of 18.0 mm, depending on the coating. Chewing gum composition (B) contains 1.5 mg nicotine per unit.

Complete composition.

| | Comp. A | Comp. B | | |
|---|---|---|---|---|
| | Quantity | Quantity | | |
| Ingredient | (mg/unit) | (mg/unit) | Function | Standard |
| Active substance | | | | |
| Nicotine | 3.30¹ | 1.65¹ | Drug substance | Ph. Eur. curr. |
| | | | | ed. |
| Gum powder PG N12 TA | 926 | | Gum base | Internal, |
| (contains maltitol) | | | | Gum Base |
| | | | | Co. S.p.A., |
| | | | | Italy |
| Gum powder: PG Nicotine | | 938 | | |
| 5TA/PG New Nik 5TA, Cool mint | | | | |
| flavour (contains sorbitol) | | | | |
| Gum powder: PG Nicotine | | 938 | | |
| 11TA/PG New Nik 11TA, Cool | | | | |
| mint flavour (contains sorbitol) | | | | |
| Microcrystalline cellulose | 121.7 | 60.85 | Nicotine carrier | Ph. Eur. curr. |
| | | | | ed. |
| Isomalt | 120.65 | 241 | Filler, sweetener | Ph. Eur. curr. |
| | | | | ed. |
| Ethanol, anhydrous | 72.0² | 59.75² | Solvent | Ph. Eur. curr. |
| | | | | ed. |
| Ascorbyl palmitate | 2.35 | 2.50 | Antioxidant | Ph. Eur. Curr. |
| | | | | Ed. |
| Acesulfame potassium | 0.500 | 0.500 | Sweetener | Ph. Eur. Curr. |
| | | | | Ed. |
| Aspartame | 0.500 | 0.500 | Sweetener | Ph. Eur. curr. |
| | | | | ed. |
| Silica | | 5.00 | Glidant | Ph. Eur. Curr. |
| | | | | Ed. |
| Core weight | 1 175 | | | |
| Coating excipients | | | | |
| Isomalt | 379.5 | 217 | Coating sugar | Ph. Eur. curr. |
| | | | | ed. |
| Purified water | 144.8² | 80.0 | Solvent | Ph. Eur. Curr. |
| | | | | Ed. |
| Ethanol (96 percent) | 18.0² | 10.9 | Solvent | Ph. Eur. curr. |
| | | | | ed. |
| Acacia | 5.60 | 3.24 | Binder | Ph. Eur. curr. |
| | | | | ed. |
| Titanium dioxide | 5.50 | 3.14 | Colouring agent | Ph. Eur. curr. |
| | | | | ed. |
| Mint liquid flavour | 5.20 | | Flavour | Internal, |
| (O.E. Menta 50/55 | | | | Muller&Koster |
| | | | | S.p.A., Italy |
| Mint liquid flavour | 1.60 | | Flavour | Internal, |
| (Evercool plus Flavour L-124397 | | | | Givaudan |
| | | | | Switzerland |
| | | | | AG |
| Aspartame | 0.271 | 0.155 | Sweetener | Ph. Eur. curr. |
| | | | | ed. |
| Acesulfame potassium | 0.271 | 0.155 | Sweetener | Ph. Eur. Curr. |
| | | | | Ed. |
| Macrogols (Macrogol 6000) | 2.10 | 1.22 | Surface polisher | Ph. Eur. curr. |
| | | | | ed. |
| Coating weight | 400 | 225 | | |
| Total weight | 1575 | 1475 | | |

¹10% overage to compensate for losses during the manufacturing process.
²Evaporates during the manufacturing process.

### Bulk Container and Closure of the Final Product

The coated chewing-gums (final product) are bulk packed in double plastic bags of polyethylene.

The final presentation is in two different packs:

i) aluminium bags, made of Transofoil® LL-OPET/polyethylene; Polyester 12 μm/Aluminium 9 μm/Polyethylene 60 μm containing 20 pieces of chewing gum, and

ii) aluminium blisters, made of PVC/PVDC-foil 250 μm/40 g/m²-20 μm standard aluminium-foil (incl. protective lacquer layer and heat seal lacquer) containing 10 pieces of chewing-gum.

Similar chewing gum compositions but with a content of 1.5 mg of nicotine were tested with respect to in vitro release employing the method described above. Four different gum powders were employed and there were minor variations in the compositions with respect to content of flavours and sweetener. The results were compared with those from Nicorette® 2 mg. The following results were obtained:

A, 1.5 mg nicotine, 11TA (n=3), Batch: 90901-0305-02

| Time (min) | Cumulative release |
|---|---|
| 0 | 0.064 |
| 2 | 0.509 |
| 5 | 0.691 |
| 10 | 0.855 |
| 20 | 0.936 |
| 30 | 0.964 |
| 45 | 1.000 |

B, 1.5 ma nicotine, New 11TA (n=3). Batch: 04C18

| Time (min) | Cumulative release |
|---|---|
| 0 | 0.055 |
| 2 | 0.685 |
| 5 | 0.795 |
| 10 | 0.884 |
| 20 | 0.932 |
| 30 | 0.959 |
| 45 | 1.000 |

C, 1.5 mg nicotine, 5TA (n=3), Batch: 90901-0305-01

| Time (min) | Cumulative release |
|---|---|
| 0 | 0.071 |
| 2 | 0.508 |
| 5 | 0.714 |
| 10 | 0.857 |
| 20 | 0.929 |
| 30 | 0.960 |
| 45 | 1.000 |

D. 1.5 ma nicotine, New 5TA (n=3), Batch: 04C29

| Time (min) | Cumulative release |
|---|---|
| 0 | 0.063 |
| 2 | 0.636 |
| 5 | 0.762 |
| 10 | 0.874 |
| 20 | 0.937 |
| 30 | 0.958 |
| 45 | 1.000 |

Nicorette® 2 mg, Batch: EF070A

| Time (min) | Cumulative release |
|---|---|
| 0 | 0.011 |
| 2 | 0.116 |
| 5 | 0.334 |
| 10 | 0.508 |
| 20 | 0.696 |
| 30 | 0.945 |
| 45 | 1.000 |

Moreover, the nicotine chewing gum composition (3 mg composition as described above; in the figures also denoted Zonnic™ 3 mg)) was compared with Nicorette® 4 mg in a bioequivalence (BE) study.

Moreover, the 1.5 mg composition and Nicorette® 2 mg were subjected to a consumer test carried out in 23 smokers. The results showed that “time to first effect”, i.e. the time it take to sense a nicotine effect after start of chewing, was about 120 seconds for the composition according to the invention, whereas it was 247 seconds for the Nicorette® composition, i.e. a clear indication that a chewing gum composition according to the invention releases nicotine much faster than Nicorette® and, moreover, that a smaller amount is required, i.e. a faster and more efficient release of nicotine from a composition of the present invention.

On a VAS scale (0-100) the subjects rated “craving for a cigarette”. For the composition of the invention (1.5 mg nicotine), the score dropped by 50 points versus 33 for Nicorette® 2 mg from minutes before administration to 10 minutes after administration, which also supports the much faster and more efficient release of nicotine from a composition of the invention compared with Nicorette®.

### Example 7

### Storage Stability of a Chewing Gum Composition Containing Maltitol

The following compositions are tested in open petri dishes at 25° C. and a relative humidity of 60% or 65% for at least week:

Compositions 1-4 of Example 1

Compositions 1-4 of Example 1, but containing maltitol instead of sorbitol Compositions A and B of Example 6

A chewing gum composition is equilibrated for one hour at the relative humidity used in the test. Then the composition is weighed and the composition is placed in an open petri dish at the stated temperature and relative humidity. Every day (same time, e.g. 10 am), the composition is weighed and the weight is recorded. After 1 week, preferably 2 weeks, the increase in weight (measure for up-take of water) should not exceed 15% for the maltitol-containing compositions.

### Example 8

### Storage Stability of a Chewing Gum Composition According to the Invention

The aim of the present example is to demonstrate an improved storage stability of a composition of the invention, i.e. a chewing gum composition containing maltitol, compared with a similar composition where sorbitol is used in stead of maltitol.

The following compositions were tested; composition I with content of sorbitol and composition II with content of maltitol:

TABLE
Development formulations of Zonnic medicated chewing-gum

| | Zonnic 1.5 mg | Zonnic 1.5 mg |
|---|---|---|
| | nicotine, | nicotine, |
| | Cool mint | Cool mint |
| | Scale-up formulation | Final formulation |
| Ingredient | Composition I | Composition II |
| Active substance | | |
| Nicotine | 1.65¹ | 1.65¹ |
| Gum powder PG Nicotine 11 | — | — |
| TA | | |
| Gum powder PG NEW NIC | 938 | — |
| 11 TA (containing sorbitol) | | |
| Gum powder PG N12 TA | — | 926 |
| (containing maltitol) | | |
| Microcrystalline cellulose | 60.85 | 123.35 |
| Isomalt | 241 | 120.65 |
| Ethanol, anhydrous | 59.75² | 54.6² |
| Talc | — | — |
| Magnesium stearate | — | — |
| Silica, colloidal anhydrous | 5.00 | — |
| Ascorbyl palmitate | 2.50 | 2.35 |
| Acesulfame potassium | 0.500 | 0.500 |
| Aspartame | 0.500 | 0.500 |
| Core weight | 1 250 | 1 175 |
| Coating excipients | | |
| Isomalt | 217 | 382.7 |
| Purified water | 80.0² | 153.4² |
| Ethanol (96 percent) | 10.9² | 19.8² |
| Acacia | 3.24 | 5.70 |
| Titanium dioxide | 3.14 | 5.60 |
| Mint liquid flavour | — | 2.60 |
| (O.E. Menta 50/55 | | |
| Mint liquid flavour | — | 1.60 |
| (Evercool plus Flavour | | |
| L-124397 | | |
| Aspartame | 0.155 | 0.277 |
| Acesulfame potassium | 0.155 | 0.277 |
| Macrogols (Macrogol 6000) | 1.22 | 1.20 |
| Coating weight (approx.) | 225 | 400 |
| Total weight | 1475 | 1575 |

¹10% overage to compensate for losses during the manufacturing process.
²Evaporates during the manufacturing process.

Composition II was packed in blister backs (two batches) and aluminium bags and composition I was packed in aluminium bags and all compositions stored at 30° C. and 65% relative humidity for stability studies.

The following results were obtained.

TABLE 8.1
Zonnic 1.5 mg nicotine medicated chewing-gum (composition II), batch no. 07B40, stored in
aluminium bags at 30° C./65% RH.

| | | | Related substances | | | | | | | |
|---|---|---|---|---|---|---|---|---|---|---|
| Storage | | | | | Nicotine- | Nicotine- | | Unknown | Sum of | |
| Months | Appearance | Assay of nicotine | Cotinine | Myosmine | cis-N-oxide | trans-N- | β-Nicotyrine | peaks single | peaks | Microbial quality |
| 0 | C | 1.56 | n.d. | 0.11 | 0.17 | 0.23 | n.d. | 0.18 | 0.69 | C |
| 3 | C | 1.60 | n.d. | 0.07 | n.d. | 0.08 | n.d. | 0.15 | 0.30 | n.a. |
| 6 | C | 1.54 | n.d. | <0.10 | <0.05 | 0.13 | n.d. | <0.05 | 0.21 | C |
| 9 | C | 1.49 | n.d. | <0.10 | 0.34 | 0.48 | n.d. | n.d. | 0.90 | n.a. |
| 12 | C | 1.58 | <0.05 | n.a. | n.d. | n.d. | n.d. | <0.05 | 0.10¹⁾ | n.a. |

C = conforms
n.d. = not detected
n.a. = not analysed
¹⁾Not including myosmine

TABLE 8.2
Zonnic 1.5 mg nicotine medicated chewing-gum (composition II), batch no. 07B40, stored in
blisters at 30° C./65% RH.

| | | | Related substances | | | | | | | |
|---|---|---|---|---|---|---|---|---|---|---|
| Storage | | | | | Nicotine- | Nicotine- | | Unknown | Sum of | |
| Months | Appearance | Assay of nicotine | Cotinine | Myosmine | cis-N-oxide | trans-N- | β-Nicotyrine | peaks single | peaks | Microbial quality |
| 0 | C | 1.56 | n.d. | 0.11 | 0.17 | 0.23 | n.d. | 0.18 | 0.69 | C |
| 3 | C | 1.53 | n.d. | 0.08 | 0.37 | 0.67 | n.d. | 0.17 | 1.49 | n.a. |
| 6 | C | 1.52 | n.d. | <0.05 | 0.43 | 0.68 | n.d. | <0.05 | 1.20 | * |
| 9 | C | 1.51 | n.d. | <0.10 | <0.05 | <0.05 | n.d. | n.d. | 0.14 | n.a. |
| 12 | C | 1.52 | <0.10 | n.a. | 0.83 | 1.43 | n.d. | <0.05 | 2.41¹⁾ | n.a. |

C = conforms
n.d. = not detected
n.a. = not analysed
* Total Viable Count Bacteria 100 Fungi n.d. E. Coli n.d.
¹⁾Not including myosmine

TABLE 8.3
1.5 mg nicotine medicated chewing-gum (composition I), stored in aluminium bags at 30° C./65%
RH.

| | | | Related substances | | | | | | | |
|---|---|---|---|---|---|---|---|---|---|---|
| Storage | | | | | Nicotine- | Nicotine- | | Unknown | Sum of | |
| Months | Appearance | Assay of nicotine | Cotinine | Myosmine | cis-N-oxide | trans-N- | β-Nicotyrine | peaks single | peaks | Microbial quality |
| 0 | C | 1.59 | n.d. | n.d. | 0.3 | 0.4 | n.d. | n.d. | 0.6 | n.a. |
| 3 | C | 1.45 | n.d. | n.d. | 1.0 | 1.5 | n.d. | 0.1 | 2.2 | n.a. |

The results clearly show an improved stability of the compositions containing maltitol compared with a sorbitol-containing composition.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Although the present invention and its advantages has been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A chewing gum composition comprising a nicotine-cellulose combination, maltitol and a gum base.

2. A chewing gum composition according to claim 1, wherein the gum base contains from 5% to 20% elastomers, from 25% to 50% w/w resins, from 20% to 40% w/w plasticizers, and from 10% to 20% water-insoluble adjuvants.

3. A chewing gum composition according to claim 1, wherein the concentration of maltitol in the composition is from 2% to 30% w/w.

4. A chewing gum composition according to claim 1, wherein maltitol is present in a concentration of from about 5% w/w to about 20% w/w.

5. A chewing gum composition according to claim 1, which is non-disintegrating.

6. A chewing gum composition according to claim 1, further comprising one or more sweeteners.

7. A chewing gum composition according to claim 6, wherein the concentration of the one or more sweeteners is from about 5% w/w to about 20% w/w (not including the content of maltitol).

8. A chewing gum composition according to claim 1 further comprising isomalt.

9. A chewing gum composition according to claim 8, wherein the concentration of isomalt is from about 5% to about 15% w/w.

10. A chewing gum composition according to claim 1 further comprising one or more antioxidants.

11. A chewing gum composition according to claim 1, wherein the composition is coated with a coating composition.

12. A chewing gum composition according to any of the preceding claims claim 1 comprising a core comprising:
i) nicotine-cellulose combination (nicotine free base and microcrystalline cellulose);
ii) a gum base;
iii) one or more sugar alcohols;
iv) one or more antioxidant agents; and
v) one or more artificial sweeteners,
the core being provided with a coating.

13. A chewing gum composition according to claim 1, wherein the gum base comprises one or more fats, waxes, emulsifiers, plasticizers, oils and flavoring agents.

14. A chewing gum composition according to claim 1, wherein the gum base is suitable for direct compression.

15. A chewing gum composition according to claim 1, wherein the chewing gum composition is prepared by direct compression.

16. A chewing gum composition according to claim 1, wherein the gum base is employed in form of one or more of Gum powder PG 11 TA, Gum powder PG 11 TA New, Gum powder PG 5 TA, Gum powder PG 5 TA New and Gum powder PG N12 TA.

17. A chewing gum composition according to claim 1, wherein the gum base is employed in powdered form and has a mean particle size of about 1 mm (as determined by sieving) or less.

18. A chewing gum composition according to claim 1, wherein the concentration of the gum base in the chewing gum composition is at the most 80% w/w.

19. A chewing gum composition according to claim 1, wherein the concentration of the gum base in the chewing gum composition is from about 25% w/w to about 80% w/w.

20. A chewing gum composition according to claim 1, wherein the concentration of nicotine-cis-N-oxide and nicotine-trans-N-oxide in the composition after storage in aluminum bags at 30° C. and 65% relative humidity for 3 months at the most is 1.5% w/w.

21. A chewing gum composition according to claim 1, wherein the concentration of nicotine-cis-N-oxide and nicotine-trans-N-oxide in the composition after storage in aluminum bags at 30° C. and 65% relative humidity for 9 months at the most is 1.5% w/w.

22. A chewing gum composition according to claim 1, wherein the concentration of nicotine-cis-N-oxide and nicotine-trans-N-oxide in the composition after storage blisters at 30° C. and 65% relative humidity for 3 months at the most is 1.5% w/w.

23. A chewing gum composition according to claim 1, wherein the weight of the composition at the most increases by 15% after storage in an open petri dish at 25° C. and a relative humidity of 60% for one week.

24. A chewing gum composition according to claim 23, wherein the increase in weight is at the most about 10%.

25. A chewing gum composition according to claim 1, wherein the weight of the composition at the most increases by 15% after storage in an open petri dish at 25° C. and a relative humidity of 60% for two weeks.

26. A chewing gum composition according to claim 25, wherein the increase in weight is at the most about 10%.

27. A chewing gum composition according to claim 1, wherein the weight of the composition at the most increases by 15% after storage in an open petri dish at 25° C. and a relative humidity of 65% for one week.

28. A chewing gum composition according to claim 27, wherein the increase in weight is at the most about 10%.

29. A chewing gum composition according to claim 1, wherein the weight of the composition at the most increases by 15% after storage in an open petri dish at 25° C. and a relative humidity of 65% for two weeks.

30. A chewing gum composition according to claim 29, wherein the increase in weight is at the most about 10%.

31. A chewing gum composition according to claim 1 for fast onset of action of nicotine.

32. A chewing gum composition according to claim 31, wherein the onset takes place within 3 minutes after application of the chewing gum composition to the oral cavity of the subject.

33. A chewing gum composition according to claim 32, wherein the application to the oral cavity includes initiation of chewing the chewing gum composition.

34. A chewing gum composition according to claim 1, which within the first 2 minutes after start of an in vitro release test (USP dissolution test, 1000 ml phosphate buffer pH 7.4, 37° C., paddle, 100 rpm) releases nicotine with a release rate corresponding to 5% w/w or more of the total content in the composition per minute.

35. A chewing gum composition according to claim 34, which—when subjected to the in vitro release test—within the first 2 minutes after start of the test releases nicotine with a release rate corresponding to 10% w/w or more of the total content in the composition per minute.

36. A chewing gum composition according to claim 34, wherein said release rate within the first 2 minutes after start of the test is 11% w/w or more of the total content of nicotine in the composition per minute.

37. A chewing gum composition according to claim 34, wherein said release rate within the first 2 minutes after start of the test is 17% w/w or of the total content of nicotine in the composition per minute.

38. A chewing gum composition according to claim 1, wherein at least 65% w/w of the total content of nicotine in the composition is released within 5 minutes when subjecting the chewing gum composition to an in vitro release test as described herein.

39. A chewing gum composition according to claim 1, wherein at least 75% w/w of the total content of nicotine in the composition is released within 10 minutes when subjecting the chewing gum composition to an in vitro release test as described herein.

40. A chewing gum composition according to claim 1, wherein onset of a nicotine effect is at the most 5 minutes after a subject has started chewing of the chewing gum composition.

41. A chewing gum composition according to claim 1, wherein the cellulose of the nicotine-cellulose combination comprises internal voids or pores or both voids and pores.

42. A chewing gum composition according to claim 41, wherein said voids or pores at least partially comprise said nicotine.

43. A chewing gum composition according to claim 1, wherein the cellulose is a cellulose derived from a plant, an algae, a bacterium, a fungi, or combinations thereof.

44. A chewing gum composition according to claim 1, wherein the cellulose has a surface area of at least 0.7 m²/g.

45. A chewing gum composition according to claim 1, wherein the cellulose in the nicotine-cellulose combination is a crystalline cellulose including a microcrystalline cellulose.

46. A chewing gum composition according to claim 1, wherein said cellulose in the nicotine-cellulose combination is a microcrystalline cellulose, which is selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof.

47. A chewing gum composition according to claim 1, wherein said cellulose in the nicotine-cellulose combination is a synthetic or semi-synthetic cellulose, or it is derived from a natural cellulose.

48. A chewing gum composition according to claim 1, wherein the mean particle size of the cellulose in the nicotine-cellulose combination is in a range of from about 15 to about 250 μm.

49. A chewing gum composition according to claim 1, wherein nicotine is at least partly sorbed on the cellulose in the nicotine-cellulose combination.

50. A chewing gum composition according to claim 1, wherein the concentration of the nicotine-cellulose combination in the chewing gum composition is at least about 2% w/w.

51. A chewing gum composition according to claim 1, wherein the concentration of the nicotine-cellulose combination in the chewing gum composition is from about 2% w/w to about 20% w/w.

52. A chewing gum composition according to claim 1, wherein the concentration of nicotine in the chewing gum composition is at least about 0.1% w/w.

53. A chewing gum composition according to claim 1, wherein the concentration of nicotine in the chewing gum composition is in a range from about 0.1% w/w to about 15% w/w as calculated as free nicotine base.

54. A chewing gum composition according to claim 1, wherein the concentration of nicotine in the chewing gum composition is from about 0.1% w/w to about 10% w/w as calculated as free nicotine base.

55. A chewing gum composition according to claim 1, wherein the chewing gum composition has a nicotine content of from about 0.5 mg to about 10 mg as calculated as free nicotine base.

56. A chewing gum composition according to claim 1, wherein the chewing gum composition comprises 1.5 mg of nicotine calculated as free nicotine base.

57. A chewing gum composition according to claim 1, wherein the chewing gum composition comprises 3 mg of nicotine calculated as free nicotine base.

58. A chewing gum composition according to claim 1, wherein the chewing gum composition comprises 5 mg of nicotine calculated as free nicotine base.

59. A chewing gum composition according to claim 1, wherein said nicotine is selected from the group consisting of nicotine base, nicotine hydrochloride, nicotine dihydrochloride, nicotine monotartrate, nicotine bitartrate, nicotine sulfate, nicotine zinc chloride such as nicotine zinc chloride monohydrate and nicotine salicylate.

60. A chewing gum composition according to claim 1, wherein nicotine in the nicotine-cellulose combination is in its free base form.

61. A chewing gum composition according to claim 1, wherein the chewing gum composition further comprises one or more buffering agents.

62. A chewing gum composition according to claim 61, wherein the one or more buffering agents is selected from the group consisting of acetates, glycinates, phosphates, glycerophosphates, citrates such as citrates of alkaline metals, carbonates, hydrogen carbonates, and borates, and mixtures thereof.

63. A chewing gum composition according to claim 61, wherein the one or more buffering agents are present in a concentration from about 0.5% w/w to about 5% w/w.

64. A chewing gum composition according to claim 6, wherein the one or more sweeteners comprise sugar alcohols or artificial sweeteners.

65. A chewing gum composition according to claim 6, wherein the concentration of the one or more sweeteners is at least about 0.05%.

66. A chewing gum composition according to claim 1, further comprising one or more anti-oxidants.

67. A chewing gum composition according to claim 66, wherein the one or more anti-oxidants are present in a concentration of from about 0.05% w/w to about 0.3% w/w.

68. A chewing gum composition according to claim 1, wherein the chewing gum composition comprises one or more flavoring agents.

69. A chewing gum composition according to claim 68, wherein the total concentration of flavoring agents in the chewing gum composition is from about 0.5% w/w to about 12% w/w.

70. A chewing gum composition according to claim 1, wherein the concentration of nicotine of a pharmaceutically acceptable salt, solvate or complex in the nicotine-cellulose combination is at the most 70% w/w, and the concentration being calculated as the nicotine base.

71. A chewing gum composition according to claim 1, wherein the concentration of nicotine in the nicotine-cellulose combination is from about 3% w/w to about 20% w/w.

72. A chewing gum composition according to claim 1, wherein the chewing gum composition comprises a pharmaceutically acceptable excipient.

73. A chewing gum composition according to claim 72, wherein the pharmaceutically acceptable excipient comprises one or more anti-adhesives, lubricants or glidants selected from the group consisting of talc, stearates and salts thereof.

74. A chewing gum composition according to claim 73, wherein the chewing gum composition comprises talc in a concentration from about 0.5% w/w to about 10% w/w.

75. A chewing gum composition according to claim 73, wherein the pharmaceutically acceptable excipient comprises magnesium stearate in a concentration from about 0.1% w/w to about 5% w/w.

76. A chewing gum composition according to claim 73, wherein the chewing gum composition comprises silica in a concentration from about 0.1% w/w to about 4% w/w.

77. A chewing gum composition according to claim 1, wherein the chewing gum composition after administration to a subject has a relative bioavailability as calculated by AUC_{0-infinity} (tested composition)/AUC_{0-infinity} (Nicorette®)×100% of at least 120% provided that the composition and Nicorette® contains the same amount of nicotine calculated as free base.

78. A chewing gum composition according to claim 1, for treatment and/or prophylaxis of nicotine addiction.

79. Use of a nicotine-cellulose combination, maltitol and a gum base for the preparation of chewing gum composition as defined in claim 1 for achievement of a fast onset of action of nicotine after application of the chewing gum composition to the oral cavity of a subject.

80. A method for preparation of a chewing gum composition defined in claim 1 comprising mixing a nicotine-cellulose combination with maltitol and a gum base and, optionally, one or more pharmaceutical acceptable excipients and forming the resulting mixture into chewing gum by direct compression.

81. A method for preparation of a chewing gum composition defined in claim 1 comprising mixing a nicotine-cellulose combination with a gum base composition comprising maltitol, and, optionally, one or more pharmaceutical acceptable excipients and forming the resulting mixture it into chewing gum by direct compression.
